(19) United States
(12) Patent Application Publication
(10) Pub. No.: US 2024/0390611 A1
(43) Pub. Date: Nov. 28, 2024

(54) ARI smart respiratory inhaler with integrated spacer

(71) Applicant: Jonathan David Piteo Tarpy, New York, NY (US)

(72) Inventor: Jonathan David Piteo Tarpy, New York, NY (US)

(21) Appl. No.: 18/201,122

(22) Filed: May 23, 2023

(51) Int. Cl. A61M 15/00

(52) U.S. Cl. CPC A61M 15/008; A61M 15/0021; A61M 15/0086; A61M 2205/3553; A61M 2205/3584; A61M 2205/8206; A61M 2230/40

(57) ABSTRACT

An ARI Smart Respiratory Inhaler with Integrated Spacer (herein referred to as a ‘smart inhaler’) is disclosed. The smart inhaler is an internet of things (IoT) inhaler that delivers medication and measures lung conditions—including partial pressure; nitric oxide; oxygen; peak flow; and forced expiratory volume. Data is collected and transmitted to a mobile app and web portal that allows patients and health care providers to track and view respiratory status in real time. Other aspects include software and a cloud network with algorithms that can predict and alert stakeholders to any trends or problems before they have a chance to manifest. The smart inhaler also leverages block chain technology to keep stakeholders informed of treatment and health status in real time. An object of the invention is to help patients maintain their medication regimen and provide health care workers with said patient's respiratory status quickly.

### CROSS-REFERENCE TO RELATED APPLICATIONS

The present application includes subject matter disclosed in and claims priority to a provisional application entitled “ARI Smart Respiratory Inhaler with Integrated Spacer” filed Jun. 5, 2022 and assigned Application Number 633,490,97 describing an invention made by the present inventor. In addition, it should be noted that the inventor also filed similar utility and design patents in Taiwan in June of 2022. These applications claim priority: TW1781673B and TWD218780S and were both filed in Taiwan and reported as per 35 U.S.C. § 119 or 365.

### FIELD OF THE INVENTION

The present invention generally relates to IoT. More specifically, it relates to a smart inhaler device.

### BACKGROUND

Internet of Things (IOT) devices first began to emerge in the early 1980s when students modified soda vending machine at Carnegie Mellon University. This machine was considered to be the first internet-connected appliance cable to reporting its inventory and informing customers when newly-loaded drinks were cold. The term IoT was most likely coined by Procter & Gamble who used radio-frequency identification tags as part of an IOT system. Others have defined IoT as simply the point in time when more ‘things or objects’ are (or will be) connected to the internet than people. Today IoT is drastically transforming how businesses are run and how society functions in general. As overall communication with IoT has started to becomes faster with the onset of 5G networks in cities and data generated from connected devices is helping businesses to run more efficiently, gain insight into business processes, and allows companies to make real-time decisions. While IoT is gaining in popularity in many industries, only a handful of IoT devices are being utilized for inhalation devices. United States Patent No. US20020032387A1 disclosed a combination spirometer and oximeter with IoT that communicates with others wirelessly—however it does not incorporate an inhaler. U.S. Pat. No. 5,522,380A disclosed a metered dose medication adaptor with an onboard spirometer-however it does not have IoT communications. U.S. Pat. No. 8,333,190B2 disclosed an IoT nebulizer with flow meter—however it does not incorporate a spirometer. United States Patent No. US20170000382A1 disclosed an IoT inhaler that tracks usage—however, it does not leverage blockchain technology. What is needed is an IoT-based, smart inhaler that can deliver inhalation medication, track usage, communicate with stakeholders and predict trends and treatment issues securely. For example, respiratory medication tracking for seniors can be especially useful—those who fail to take medications on a regular basis can experience a variety of problems. First, they may not receive the full therapeutic benefits of the medications they have been prescribed. This can lead to a worsening of existing health conditions or even new health problems. Second, failure to take medication regularly can lead to complications such as hospitalization, prolonged illness, and increased healthcare costs. When seniors miss doses, it can be challenging for healthcare professionals to manage their conditions effectively and keep them healthy. Third, the inability to adhere to medication schedules can often signal other health problems, such as memory loss or depression, which may require additional treatment or support. Therefore, it is essential for seniors to take their medications regularly and follow the prescribed schedule. The smart inhaler can play a crucial role in helping seniors to manage their medications effectively, which can help improve their health outcomes and overall quality of life.

### SUMMARY OF THE INVENTION

The device herein disclosed and described provides a solution to the shortcomings in the prior art through the disclosure of an IoT-based, smart inhaler. An object of the device is to deliver respiratory medicine to a patients' lungs. The device includes an inhaler with adapter to receive disposable, conventional, pressurized, vapor cannisters, powder cartridges and is used in typical manner (the patient inserts it into their mouth and presses a button to release the vapor medication into the lungs). For this disclosure, a conventional inhaler is defined as: a medical device that delivers medication directly to the lungs in the form of a mist or spray. Inhalers are commonly used to treat respiratory conditions such as asthma, chronic obstructive pulmonary disease, and bronchitis. They typically consist of a small canister of medication and a mouthpiece, and work by delivering a measured dose of medication when the user inhales through the mouthpiece. Inhalers are designed to be portable and easy to use, and are often used to manage symptoms of respiratory conditions on a daily basis. In this disclosure, (IoT) refers to the network of physical objects and devices that are embedded with sensors, software, and connectivity, allowing them to collect and exchange data over the internet. Another object of the invention is to minimize medication deposits in the inhaler and maximize delivery deep into the lungs by means of a novel integrated spacer having a converging-diverging nozzle. This unique chamber spacer relies upon an air intake which causes the dispersed medication to be mixed with air when inhaled by the user through a converging-diverging airflow pathway. The resulting mixture in the medication molecules are slowed down for easier inhalation, and the harmful carrier molecules are then evaporated off so as not to be inhaled by the user or deposited in the mouth and throat.

Another object of the invention is to provide onboard sensing technologies to measure lung functions and parameters that include but are not limited to: partial pressure oxygen; nitric oxide; peak flow; and forced expiratory volume. Such data can provide an overall status of a patient's overall lung health. Such data can be extremely useful when monitoring patients who have complex, respiratory illnesses such as COVID19, and pneumonia etc. The device also has onboard exhalation body temperature sensor—this sensor allows the system to track changes in inflammation that is correlated with lung diseases such as Asthma and COPD etc. For this disclosure, the onboard sensing is considered to be ‘micro sensors’ which for this disclosure are defined as tiny electronic devices that are designed to detect and measure physical or chemical properties of their environment, such as temperature, pressure, humidity, light, or gas concentration. They typically consist of a sensing element and an electronic circuit that converts the sensed information into an electrical signal that can be analyzed or transmitted to other devices. Micro sensors are usually fabricated using microelectromechanical systems technology, which allows for the production of small, lightweight, and low-power sensors that can be integrated into a wide range of applications, including biomedical devices, environmental monitoring systems, and industrial control systems.

Another object of the invention is to allow all device data to be collected and transmitted wirelessly via Bluetooth to a mobile device app and then to a web portal that allows patients and their health care providers to track and view their respiratory health status in real time. This function can help vulnerable patients such as elderly who may not always track or report on their conditions to their physicians. In this disclosure, wireless is defined as the transmission of data or information between two or more devices without the use of physical cables or wires. Instead, wireless communication uses electromagnetic waves, such as radio waves, to carry information from one device to another. Examples of wireless communication technologies include Wi-Fi, Bluetooth, cellular networks, and satellite communication.

Another object of the invention is to include software and a cloud network with algorithms that can predict and alert stakeholders to any trends or problems before they have a chance to manifest. Such algorithms may include artificial intelligence routines for predictive analysis. Data from the system is fed to algorithms in the cloud network that analyze it and compare conditions to a database that allows for predictions regarding any trends. For example, an asthmatic patient is reported to have an onset of low oxygen and low forced expiratory volume and isn't feeling well. The algorithm can predict a problem and notify the patient to seek medical treatment in the near future. For this disclosure, a cloud network is defined as a type of computer network that uses cloud computing technology to provide services and resources over the internet. In a cloud network, data and applications are hosted on servers located in remote data centers, and users access them through the internet. Cloud networks typically provide on-demand access to scalable computing resources, such as processing power, storage, and network bandwidth, which can be adjusted dynamically to meet changing user needs. Cloud networks are often used by businesses and organizations to reduce their IT infrastructure costs, improve their scalability and flexibility, and enhance their data security and disaster recovery capabilities.

In this disclosure, artificial intelligence is defined as a field of computer science that involves the development of intelligent machines that can perform tasks that typically require human intelligence, such as visual perception, speech recognition, decision-making, and language translation. AI technologies use algorithms and mathematical models to analyze large amounts of data and learn from patterns and trends, in order to improve their performance over time. Examples of AI applications include chatbots, autonomous vehicles, image and speech recognition systems, recommendation engines, and fraud detection systems. AI is considered to be a rapidly growing and transformative technology that has the potential to significantly impact a wide range of industries and domains.

Another object of the invention is to leverage the security of block chain technology to provide a historical database and keep a multitude of stakeholders informed of all smart respirator activity, sensors, treatments and patient health status in real time over a decentralized ledger based on smart contracts. For this disclosure, blockchain is defined as a decentralized digital ledger technology that allows multiple parties to store, share and validate data in a secure and transparent manner. In a blockchain system, data is stored in blocks that are linked together in a chronological and immutable chain, using cryptographic techniques to ensure the integrity and authenticity of the data. Each block contains a unique digital signature, called a hash, which is generated based on the data in the block and the hash of the previous block in the chain. This makes it virtually impossible to modify or tamper with the data stored in the blockchain without being detected.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

The accompanying drawings, which are incorporated herein and form a part of the specification, illustrate some, but not the only or exclusive, examples of embodiments and/or features.

FIG. 1 shows a top perspective view of the smart inhaler.

FIG. 2 shows an exploded view of the smart inhaler.

FIG. 3 shows a representative view of the smart inhaler functions.

FIG. 4 shows a representative view of the block chain.

FIG. 5 shows a representative view of the smart inhaler method.

Other aspects of the present invention shall be more readily understood when considered in conjunction with the accompanying drawings, and the following detailed description, neither of which should be considered limiting.

### DETAILED DESCRIPTION OF FIGURES

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation. Conventional components of the invention are elements that are well-known in the prior art and will not be discussed in detail for this disclosure. smart inhaler

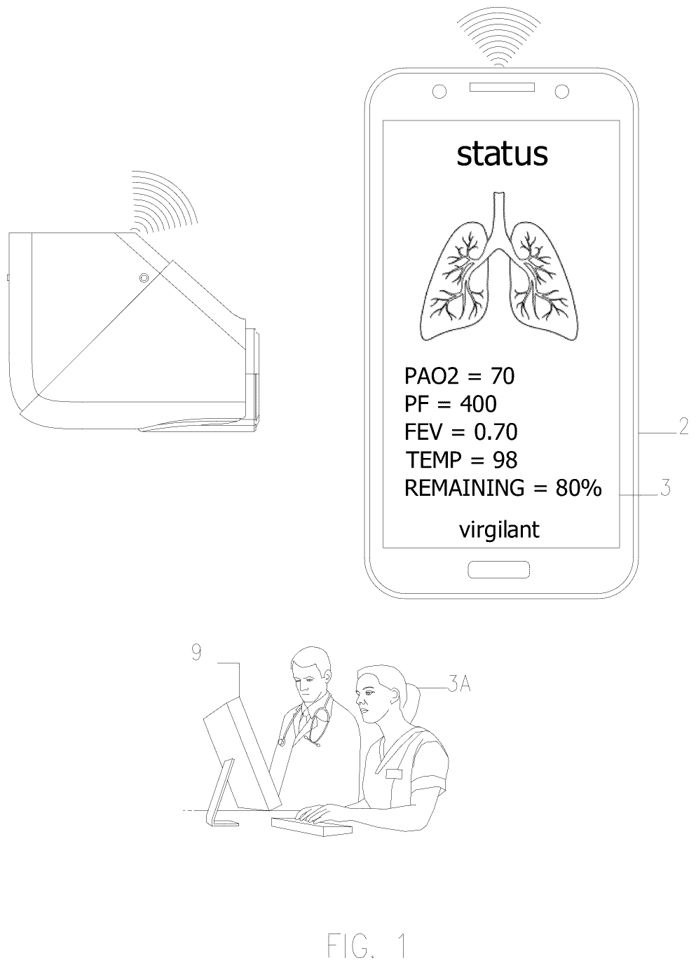

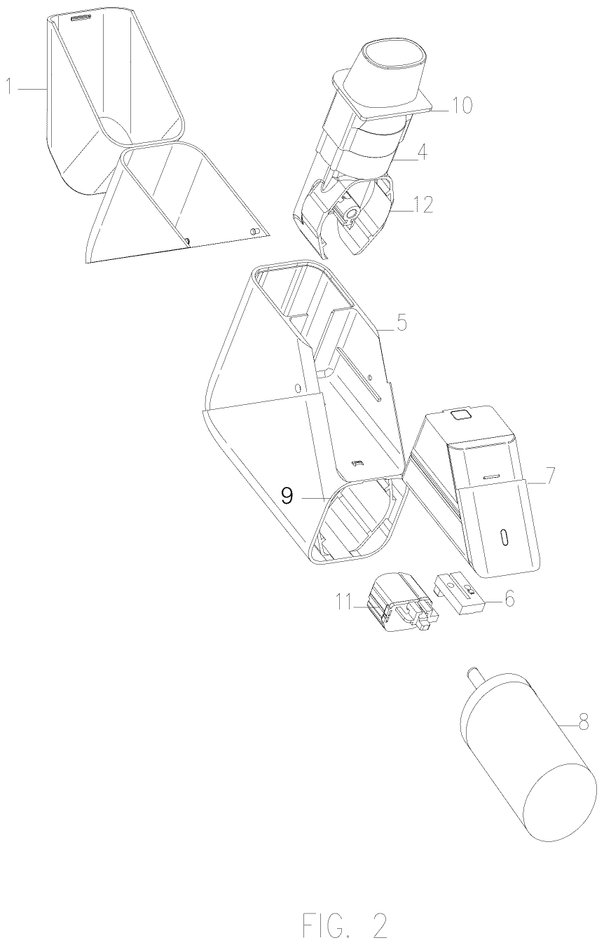

FIG. 1 shows a perspective view of a preferred embodiment of the smart inhaler having a triangular-shape and communicating with mobile software application 3 (app) on a mobile phone 2 as well as a desktop computer 9 being operated by health care providers 3A. Said app being on a non-transitory computer readable medium including computer readable instructions. FIG. 2 showing an exploded view of the smart inhaler 1 having components that include but are not limited to: outer casings 1 and 5.; mouthpiece 10; onboard battery 11 (such as lithium ion and the like); vapor accelerator 12 with attachment to receive conventional medicine cannister 8 nozzle therein; integrated spacer 4 for receiving and securing body of said medicine cannister 8; square-shaped liner 7 configured to receive microcontroller 6 therein. Said integrated spacer 4 is a converging-diverging nozzle providing enhanced medicine delivery and flow-specifically, it increases the mixing of propellant and inhalation air while avoiding the deposition of the drug on inner parts of the device. The integrated spacer 4 prevents the deposition of the drug in the throat while maintaining respirable fraction performance. In addition, integrated spacer 4 prevents the deposition of medication in the throat thereby allowing it to reach deep into the lungs. The smart inhaler also having removable medicine cannister 8 held into outer casing 5 by means of guide 9. All of the aforementioned components (with the exception of microcontroller 6 and medicine cannister 8) are made of a rigid, lightweight material such as plastic and the like. Said microcontroller 6 having onboard memory with proprietary firmware that governs a plurality of onboard sensors to detect parameters that include but are not limited to: lung functions, partial pressure oxygen; nitric oxide; exhalation body temperature, peak flow; and forced expiratory volume, temperature, medication remaining flow rates, and temperature etc. Microcontroller 6 also having an onboard wireless communications module (Bluetooth etc.) for data transmission.

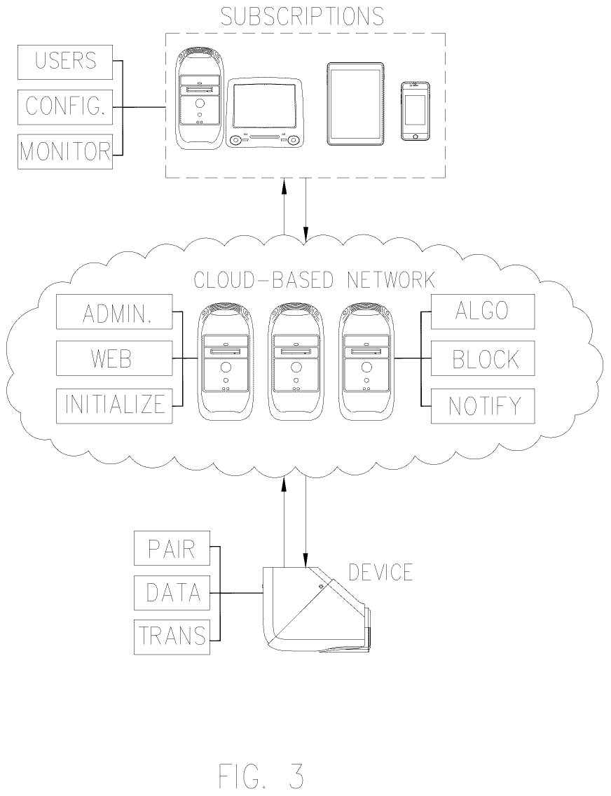

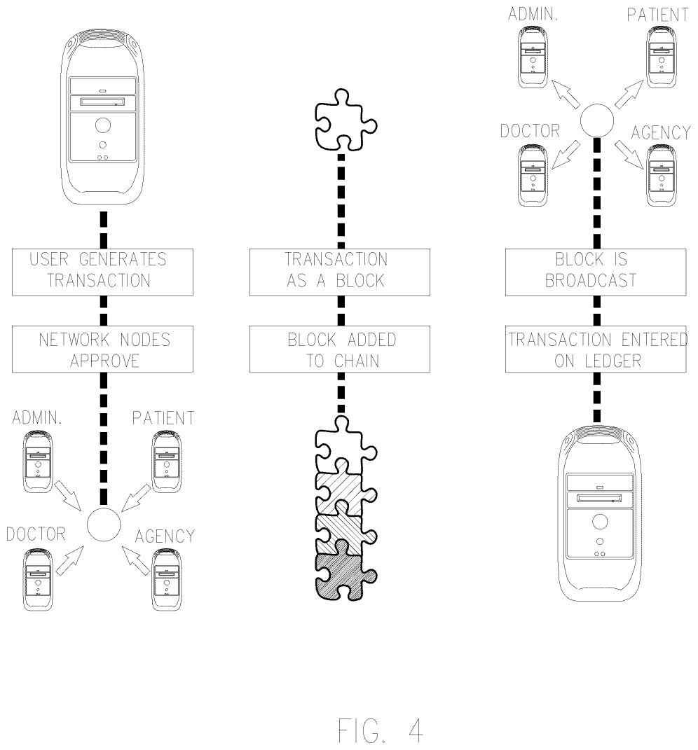

FIG. 3 shows a representative view of the cloud-based network wherein users have functions that include but are not limited to: software subscription level choice; the ability to pair and configure new and existing smart inhalers and the ability to monitor data and readings on a computing device such as but not limited to: desktop computer, laptop, tablet and mobile phone etc. The smart inhaler having functions including but not limited to: pairing with another wireless device; collecting and receiving patient data and transmitting data wirelessly. The user and the smart inhaler being connected by means of a cloud-based network with operations that include but are not limited to: administrative routines (patient profiles, demographics and subscription management); web portal with logins for stakeholders and users to view all smart inhaler sensor data in real time; initializations (pairing new inhalers, configuring thresholds and calibrations); artificial intelligence algorithms for trend detection and condition predictions; encrypted data collection stored on a block chain under a decentralized ledger for all stakeholders; and automated notifications to stakeholders when any sensor thresholds are breached or extreme trend detections are observed (via text messaging, SMS, email etc.). FIG. 4 shows a representative view of the block chain software whose main function is building a non-fungible, patient record that allows the patient to be compensated for use of their data and an adherence to care plan recognizing an initial transaction (sensor reading etc.). The figure shows the block chain with registered genesis block after being approved by network nodes made up of but not limited to: administrators, patients, doctors, insurance agencies and the like. When a patient continues transactions including registration, pairing devices, each transaction being is encrypted and added to the previous block forming a hashed block chain wherein each transaction has a timestamp and metadata that is broadcast to said authorized stakeholders as a recorded transaction and being available only on the cloud-based network. By design, a block chain is resistant to modification of the data. For use as a distributed ledger, the block chain is typically managed by a peer-to-peer network, collectively adhering to a protocol for inter-node communication and validating new blocks. Once recorded, the data in any given block cannot be altered retroactively, without alteration of all subsequent blocks, which requires consensus of the network majority. Combined with smart contracts, such block chains can be considered as a decentralized notary service that allows for transparency so that anyone with preset privileges can see what is inside a data element record constructed using cryptographic hash and time stamped. Smart contracts are dynamic, live contracts that once created cannot be changed but can perform certain actions when certain conditions are met-such as sharing records automatically with pre-approved parties using digital signatures. The figure also showing and embodiment of the notification process as patients, administrators, doctors and agents are notified of said patient's smart inhaler data posted on a website on the cloud interface as well as on smart phones and desktop computers.

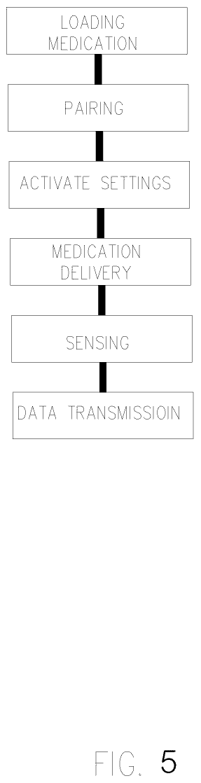

FIG. 5 shows a representative view of the smart inhaler's method that includes but is not limited to the following steps: a user loading a conventional medicine cartridge inside the device; pairing the smart inhaler(s) wirelessly with the app and/or web portal; activate settings (sensor types, sensor thresholds, dose schedules and alarm settings); medication delivery to the user; sensors detecting parameters; and delivering sensor data to the cloud network.

In view of the disclosure provided herein, a mobile application is created by techniques known to those of skill in the art using hardware, languages, and development environments known to the art. Those of skill in the art will recognize that mobile applications are written in several languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof. The software also compatible with a plurality of operating systems such as, but not limited to: Windows™, Apple™, and Android™, and compatible with a multitude of hardware platforms such as, but not limited to: personal desktops, laptops, tablets, smartphones and the like. Suitable mobile application development environments are available from several sources. Commercially available development environments include, by way of non-limiting examples, AirplaySDK, alcheMo, Appcelerator®, Celsius, Bedrock, Flash Lite,.NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK. Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications. In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types.

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are n one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth.

What is claimed is:

1. A system for delivering medicine and tracking respiratory health of a patient comprising the following parts:
a) an inhaler for delivering respiratory medication;
b) microcontroller for governing sensors;
c) a web portal for accessing a cloud network; and
d) a mobile software application;

2. The system for delivering medicine and tracking respiratory health of a patient of claim 1, wherein the inhaler having outer casings with guides.

3. The system for delivering medicine and tracking respiratory health of a patient of claim 1, wherein the inhaler having a vapor accelerator for receiving medicine cannister nozzle.

4. The system for delivering medicine and tracking respiratory health of a patient of claim 1, wherein the inhaler having a mouthpiece.

5. The system for delivering medicine and tracking respiratory health of a patient of claim 1, wherein the inhaler having a liner.

6. The system for delivering medicine and tracking respiratory health of a patient of claim 1, wherein the inhaler having an integrated spacer for enhancing medication delivery.

7. The system for delivering medicine and tracking respiratory health of a patient of claim 6, wherein the integrated spacer having a converging-diverging nozzle for slowing medication molecules.

8. The system for delivering medicine and tracking respiratory health of a patient of claim 1, wherein the mobile software app being a non-transitory computer readable medium including computer readable instructions.

9. The system for delivering medicine and tracking respiratory health of a patient of claim 1, wherein the inhaler also includes an onboard battery.

10. The system for delivering medicine and tracking respiratory health of a patient of claim 1, wherein the microcontroller having a wireless transmitter, onboard memory, and firmware for governing sensors.

11. The system for delivering medicine and tracking respiratory health of a patient of claim 1, wherein the sensors include those that measure temperature, partial pressure; nitric oxide; oxygen; peak flow; and
forced expiratory volume.

12. The system for delivering medicine and tracking respiratory health of a patient of claim 1, wherein the software app tracking respiratory status in real time.

13. The system for delivering medicine and tracking respiratory health of a patient of claim 1, wherein the web portal for accessing a cloud network also having functions that include administrative routines, web portal with logins for stakeholders, initializations, artificial intelligence algorithms for trend detection and condition predictions; and encrypted data collection stored on a block chain, and automated notifications for stakeholders.
